(19) United States
(12) Patent Application Publication
(10) Pub. No.: US 2025/0377467 A1
(43) Pub. Date: Dec. 11, 2025

(54) ELECTRONIC CIRCUIT, PHOTON COUNTING X-RAY DETECTOR, COMPUTED TOMOGRAPHY SYSTEM AND METHOD FOR CAPTURING COINCIDENCE EVENTS OF A COMPUTED TOMOGRAPHY SYSTEM

(71) Applicant: Siemens Healthineers AG, Forchheim (DE)

(72) Inventors: Michael HOSEMANN, Erlangen (DE); Edgar GOEDERER, Forchheim (DE); Pascal RAFFELBERG, Duisburg (DE)

(73) Assignee: Siemens Healthineers AG, Forchheim (DE)

(21) Appl. No.: 19/228,835

(22) Filed: Jun. 5, 2025

(30) Foreign Application Priority Data
Jun. 7, 2024 (EP) 24180792.4

(51) Int. Cl. G01T 1/24; H04N 25/30

(52) U.S. Cl. CPC G01T 1/247; H04N 25/30

(57) ABSTRACT

An electronic circuit for capturing coincidence events includes a detector pixel array, the circuit including a first capturing unit which is configured to provide a first impingement event signal. For each further detector pixel of at least one further detector pixel of the detector pixel array, the electronic circuit includes a further capturing unit configured to provide a further impingement event signal. The electronic circuit includes a logic circuit which compares the first impingement event signal with the at least one further impingement event signal and provides a coincidence signal based on a result of the comparison. A coincidence counter of the electronic circuit increments a coincidence count of the coincidence counter based on the coincidence signal. For each further detector pixel of the at least one further detector pixel, the electronic circuit includes a switching unit configured to disconnect the respective further impingement event signal.

### CROSS-REFERENCE TO RELATED APPLICATION(S)

The present application claims priority under 35 U.S.C. § 119 to European Patent Application No. 24180792.4, filed Jun. 7, 2024, the entire contents of which are incorporated herein by reference.

### FIELD

One or more example embodiments relates to an electronic circuit, a method for capturing coincidence events of a computed tomography system, to a photon counting X-ray detector with such an electronic circuit and a computed tomography system with such an electronic circuit. The computed tomography system includes a photon counting X-ray detector with a detector pixel array.

### RELATED ART

While incident X-ray photons are firstly converted into optical photons (scintillation) in conventional detectors for computed tomography systems (hereinafter CT systems), photon counting X-ray detectors convert the X-ray photon on the active surface directly into an electrical signal (direct conversion). Positive and negative electrical charges, which are generated by incident X-ray photons, are separated by electrical fields and the charge quantity, and consequently the energy of the X-ray photon, is reproduced by the amplitude of the electrical signal. As a rule, successive events overlap due to the high signal duration in the case of scintillation detectors, which lies in the region of microseconds. For this reason, these detectors are also referred to as energy-integrating detectors. If semiconductor detectors (for example based on silicon, cadmium telluride, cadmium zinc telluride or gallium arsenide) which can be read out very quickly, for example in the region of nanoseconds, are used instead, individual photons can be counted and can be sorted, for example with the aid of a comparator, into a histogram. This technique makes a spectral separation and thereby improved subsequent material differentiation possible. The electrical noise in the detector is reduced by circumventing the indirect conversion, so improved signal-to-noise ratios are achieved or the same signal-to-noise ratio with a lower dose.

X-ray detectors in CT systems can have, for example, a number of detector pixels of greater than 1 million. Furthermore, CT systems can possibly also have a plurality of such X-ray detectors.

The simultaneous occurrence of at least two count events is referred to as a coincidence event in connection with CT systems, which events are each identified at a corresponding detector pixel of the photon counting X-ray detector. For example, these count events can be identified in detector pixels which are in close proximity. When an X-ray photon impinges on the boundary surface of two abutting detector pixels, it is possible, for example, that the energy of the X-ray photon is identified at two or more detector pixels. It is also possible that secondary photons are released by the impinging of the X-ray photon on a detector pixel, and these are then identified by another detector pixel. This can result in image errors of the reconstructed image. To prevent or reduce such errors, photon counting CT systems can include electronic circuits to identify the coincidence events.

These electronic circuits can identify, for example, whether impingement events simultaneously occur in a defined neighborhood in the detector pixel and at least one further detector pixel. For this, for example signal lines, hereinafter also referred to as coincidence lines, are installed between the detector pixels which are involved. The information about the impingement in one detector pixel can then in each case be transferred via these coincidence lines to the corresponding remainder of the detector pixel which are involved. If, for example, coincidence events from a 4-neighborhood of detector pixels are taken into account, eight coincidence lines per detector pixel can result due to this line routing. In other solutions, even more coincidence lines can possibly be necessary.

Lines for transmitting digital signals in the form of pulses can have parasitic resistances and parasitic capacitances. Lines can form arrangements between wires of the same line and/or between the wires and the line surroundings, which arrangements act in a manner similar to a capacitor. The electrical fields which occur can store energy and thus form a capacitor. Depending on the length of the line, pulse shape and frequency, etc., the electrical field or this capacitor can have different sizes. An increased power requirement can result in the event of signal changes due to the necessary reloading of the capacitors.

### SUMMARY

With a large number of lines between the individual detector pixels, the increased power requirement of the relevant electronic circuits of the CT system accumulates and/or sensitive analog circuits in the surrounding area are affected due to crosstalk between lines.

One or more example embodiments reduces the adverse effects of the coincidence lines on the CT system.

This is achieved by the respective subject matter of the independent claims. Advantageous developments and preferred embodiments are the subject matter of the dependent claims, the following description as well as the figures.

One or more example embodiments is based on the finding that the coincidence identification is not required or used in every application and/or depending on application, is required or used in a different manifestation. Therefore, the optional decoupling from a further detector pixel, which is to be taken into account for coincidence identification, is made possible for a given detector pixel.

### BRIEF DESCRIPTION OF THE DRAWINGS

Example embodiments will be explained in more detail below using associated schematic drawings. In the figures, identical or functionally identical elements can be provided with the same reference characters. The description of identical or functionally identical elements will possibly not necessarily be repeated in respect of different figures.

In the drawings:

FIG. 1 shows a schematic block diagram of an exemplary embodiment of an inventive electronic circuit for capturing coincidence events of a computed tomography system;

FIG. 2 shows a schematic block diagram of a further exemplary embodiment of an inventive electronic circuit;

FIG. 3 shows a schematic block diagram of a further exemplary embodiment of an inventive electronic circuit;

FIG. 4 shows a schematic block diagram of a further exemplary embodiment of an inventive electronic circuit; and

FIG. 5 shows a schematic block diagram of a logic circuit of a further exemplary embodiment of an inventive electronic circuit.

### DETAILED DESCRIPTION

According to one or more example embodiments, an electronic circuit for capturing coincidence events of a CT system is disclosed. This system has a photon counting X-ray detector which includes a detector pixel array. The electronic circuit includes a first capturing unit which is configured to provide a first impingement event signal as a function of an energy captured via a detector pixel of the detector pixel array. In addition, for each further detector pixel of at least one further detector pixel of the detector pixel array, the electronic circuit includes a further capturing unit which is configured to provide a further impingement event signal as a function of an energy captured via the respective further detector pixel. In addition, the electronic circuit includes a logic circuit which is configured to compare the first impingement event signal with the at least one further impingement event signal and to provide a coincidence signal as a function of a result of the comparison. In addition, the electronic circuit includes a coincidence counter which is configured to increment a coincidence count of the coincidence counter as a function of the coincidence signal. In addition, for each further detector pixel of the at least one further detector pixel, the electronic circuit includes a switching unit which is configured to disconnect the respective further impingement event signal from the logic circuit.

With a photon counting CT system, the incident X-ray photons can generate free charge carriers (electrons and holes) on the active surface of the detector pixel and these can be separated with the aid of an electrical field. The charges are separated, for example, in a strong electrical field between a cathode at the upper side and at least one anode at the lower side of the detector pixel. The charge can be represented, for example with the aid of a detector circuit of the detector pixel, as an analog impingement event signal. The measured charge corresponds to the electrical energy of the X-ray photon and can thereby be an indicator of the impingement of an X-ray photon on the active surface of the detector pixel.

The first capturing unit and the at least one further capturing unit can convert the respective impingement event signal into a digital pulse which can mark the information about an impingement event for the respective detector pixel. The respective impingement event signal is provided, in particular, at an output of the corresponding capturing unit. A measured charge can also develop owing to different effects if a photon only partially impinges on the active surface or does not impinge at all. A transmission of such charges as digital pulses can be referred to as a false-positive event or also as a coincidence event and can result in image errors.

To identify coincidence events, the logic circuit can compare the first impingement event signal of the detector pixel with the at least one further impingement event signal of the at least one further detector pixel. If the first impingement event signal has a pulse and at the same instant one of the at least one further impingement event signals likewise has a pulse, the logic circuit can detect this as a coincidence event and indicate it at its output as a digital pulse. In other words, the output of the logic circuit can remain, for example, at a logical zero as long as no coincidence signal is identified. A logical one at the output of the logic circuit then symbolizes, for example, a coincidence event.

The coincidence counter can capture the coincidence events, for example, for a fixed readout period. For this, the coincidence count, which initializes at the start of the readout period, that is to say was set to zero, can be incremented by one respectively as soon as a pulse is detected in the coincidence signal.

The at least one switching unit can be configured, for example, optionally for connecting or disconnecting the connection of the at least one further impingement event signal to/from the logic circuit. In other words, the respective switching unit can connect the respective further impingement event signal either to the logic circuit, for example in a first operating mode of the CT system, or disconnect the connection, for example in a second operating mode of the CT system. In particular for the case where the number of the at least one further detector pixel is greater than one, the respective switching unit can be individually switched on or off. Therefore, each further impingement event signal of the at least one further impingement event signal can be optionally connected to the logic circuit or be disconnected from the logic circuit independently of one another.

In the case where the number of further detector pixels is greater than one, the logic circuit can, for example, compare the respective further impingement event signals firstly among themselves and subsequently compare an intermediate result of this first comparison with the first impingement event signal in a second comparison. Similarly, the logic circuit can, for example, firstly compare the first impingement event signal with each further impingement event signal of the at least one further impingement event signal in a first comparison and subsequently carry out a second comparison of the respective intermediate results.

In this case and below, a comparison of signals can be understood, for example, in such a way that a logic gate or an interconnection of logic gates are provided at the input side with the signals which are to be compared and a result of the comparison is obtained at the output side from the logic gate or the interconnection of logic gates.

For example, for each further impingement event signal of the at least one further impingement event signal, the at least one switching unit can include a switching unit which is in each case configured to optionally disconnect the respective further impingement event signal from the logic circuit or to connect the signal to it. For example, for each further impingement event signal of the at least one further impingement event signal, the logic circuit can have an input which is connected to the output of the corresponding further capturing unit, with the respective switching unit being arranged in the respective connection.

In particular, the detector pixel can have an active surface which can capture the energy of the X-ray photons incident on the corresponding detector pixel. This can similarly apply analogously to the at least one further detector pixel. Apart from the respective active surface, both the detector pixel as well as the at least one further detector pixel can also have further electronic components, in particular components of the inventive electronic circuit. The electronic circuit can also be distributed among a plurality of detector pixels, in particular among the detector pixel, the at least one further detector pixel and/or among at least one further detector pixel again. The electronic circuit can also be provided wholly or partially outside of the detector pixel array, for example inside other components of the CT system or as an external electronic circuit.

The inventive electronic circuit makes it possible to individually reduce the parasitic capacitance, induced by the circuit connections for the coincidence identification, as needed. Owing to different influencing factors it can be advantageous or necessary within an examination with a CT system for the coincidence identification to be switched off for individual detector pixels or for the entire X-ray detector. It is also possible that with one detector pixel, not all but only some of the further detector pixels are excluded from the coincidence identification. The influencing of sensitive analog circuits in the surrounding area can be reduced by the inventive electronic circuit and reactive power can likewise be saved. The inventive electronic circuit can likewise also be advantageous for targeted testing of the electronic circuit during production, for example the production of an integrated semiconductor circuit, or in system tests.

The described connection between the respective further impingement event signal and the logic circuit can, in particular, also be reciprocally embodied, in other words the first impingement event signal can be connected to one further logic circuit respectively of the at least one further detector pixel via a respective further switching unit. This arrangement produces one switchable transmission line respectively and one switchable receiving line respectively between the detector pixel and the at least one further detector pixel.

According to at least one embodiment, the first capturing unit includes a comparator and/or the at least one further capturing unit includes one further comparator respectively.

With a photon counting CT system, the incident X-ray photons on the active surface of the detector pixel can free charge carriers (electrons and holes) which are separated with the aid of an electrical field. The charges are separated in at least one embodiment in a strong electrical field between a cathode at the upper side and at least one anode at the lower side of the detector pixel. The charge can be ascertained, for example with the aid of a detector circuit of the detector pixel, as an analog detector signal. The comparator is connected to the analog detector signal of the detector pixel and receives it at the input side. The respective further comparator is connected to the analog detector signal of the respective further detector pixel and receives it at the input side.

Above a certain charge, for example, an impingement or partial impingement of an X-ray photon on an active surface of the detector pixel can be assumed. This charge can be stored, for example, as a predefined threshold value in the comparator or a predefined further threshold value in the further comparator. However, the predefined threshold value or the predefined further threshold value can also correspond to a higher energy, in particular if a plurality of comparators with different predefined threshold values are provided, and this can also be referred to as spectrally resolved counting.

For the electronic circuit, the comparator represents the complete and rapid capture of the relevant events in relation to the image representation of the computed tomography system. The comparator and the further comparator make the transition from an analog detector signal, which corresponds to a charge, into a digital detector signal possible, and thereby the advantageous representation in a histogram. The simple further processing of the impingement events of X-ray photons on the active surface of the detector pixel is one advantage of the use of comparators.

According to at least one embodiment, the detector pixel and each further detector pixel of the at least one further detector pixel abut one another.

In other words, two detector pixels, which abut one another, have a shared boundary. This neighborhood of pixels which abut one another in this way is referred to as 4-neighborhood in digital image processing since each pixel, which is not a boundary pixel, then has four neighboring pixels. In this case, the number of the at least one further detector pixel is, for example, equal to four, or three in the case of a boundary pixel, which is not a corner pixel, and two in the case of a corner pixel. The shared boundaries increase the probability of a coincidence event and are deemed particularly relevant when it is a matter of preventing errors owing to coincidence events.

According to at least one embodiment, each further detector pixel of the at least one further detector pixel is located in a predefined neighborhood of the detector pixel.

In digital image processing, a neighborhood designates a defined image region around a detector pixel. In the case of the 8-neighborhood, a second exemplary neighborhood exists which can be considered. With this neighborhood, the detector pixels which are diagonal respectively in relation to the 4-neighborhood are considered, which pixels respectively abut a corner of the detector pixel. Furthermore, other neighborhood relationships between detector pixels which could be relevant in specific applications are also conceivable.

In particular, the case of the boundary problem is also considered. As soon as a detector pixel is located at the boundary of the X-ray detector, for example a complete 4-neighborhood or a complete 8-neighborhood is possibly no longer available. In this case, for example only the coincidence signals of the detector pixels are considered which are available in this boundary region.

Electronic circuits for identifying the coincidence events can be based on knowledge of each detector pixel from identifications of its respective direct neighboring pixels. Direct neighboring pixels should hereinafter be taken to mean the detector pixels within a 4-neighborhood. Each detector pixel has four, two horizontal and two vertical, direct neighbors. These direct neighboring pixels are characterized in that they share one pixel boundary respectively with the detector pixel. They are referred to as 4-neighbors.

This embodiment has the advantage that precisely those detector pixels can be included in the coincidence consideration or can be individually disconnected by the respective switching unit, which are most relevant to this consideration. Different numbers of at least one further detector pixel and thereby different numbers of transmission lines, receiving units and switching units result depending on the selected neighborhood.

According to at least one embodiment, the electronic circuit has a control circuit which is configured to control a switching state of the respective switching unit.

In other words, the control circuit can actuate the respective switching unit to connect the respective further impingement event signal either to the logic circuit or to cut the connection, for example depending on the operating mode of the CT system. The control circuit can be configured as a central unit for this purpose, i.e. as a central controller for a plurality of detector pixels or for all detector pixels of the X-ray detector, for example. However, a modular control circuit is also possible which is configured, for example, to control a few detector pixels or one detector pixel of the X-ray detector.

One advantage of the control circuit is the central access to the at least one switching unit and thereby to the connection between the at least one further impingement event signal and the logic circuit. A fast and coordinated configuration of the CT system is thereby possible.

According to at least one embodiment, the respective switching unit includes a multiplexer which is configured to provide the logic circuit with an alternative signal as an alternative to the respective further impingement event signal.

A multiplexer is a selection circuit in analog and digital electronics, with which, in particular from a number of input signals, one signal is selected and can be connected through to the output of the multiplexer. For example, the respective Multiplexer can switch over between the respective further impingement event signal and the respective alternative signal. A first input of the multiplexer is connected, in particular, to the respective further impingement event signal and a second input of the multiplexer is connected, in particular, to the respective alternative signal. If present, at least one further input of the multiplexer can also be unconnected, that is to say without a defined reference potential.

As already described above, this embodiment can also be reciprocally constructed, that is to say that the further switching unit likewise includes a multiplexer in some embodiments, which is configured to provide at least one first alternative signal as an alternative to the first impingement event signal.

One advantage of these embodiments is that the possibilities for signal routing can be flexibly designed. Depending on configuration, the respective multiplexer can be configured to transmit the respectively necessary signals to the logic circuit.

According to at least one embodiment, the respective switching unit is configured to provide the logic circuit with an alternative signal or a constant reference potential respectively as an alternative to the respective further impingement event signal.

In other words, the respective switching unit can provide the logic circuit with, for example, a ground potential or a predefined other voltage potential as an alternative to the respective further impingement event signal. For this, the respective switching unit can disconnect the respective further impingement event signal from the logic circuit. This alternative switches off the coincidence identification for the corresponding connection. In addition, the at least one switching unit can connect the constant reference potential to the logic circuit.

In the event of use of a multiplexer as described, the at least one alternative signal can correspond to the constant reference potential.

One advantage of these embodiments is that the corresponding line in the switching state with the constant reference potential has, for example, no pulses and thereby the conducted parasitic capacitance of the line can be reduced.

According to at least one embodiment, the electronic circuit includes a second capturing unit which is configured to provide a second impingement event signal as a function of the energy captured via the detector pixel, and the electronic circuit includes an impingement event counter which is configured to increment a count of the impingement event counter as a function of the second impingement event signal.

In other words, a second capturing unit is disclosed for the detector pixel, which can evaluate the energy captured via the detector pixel. The second capturing unit can, in particular, include a second comparator which is configured with a second predefined threshold value. This second predefined threshold value can be identical to or also different from the predefined threshold value. A redundancy or a further spectral resolution may thus be achieved.

The impingement event counter can have the same mode of operation as the coincidence counter. The impingement event counter can capture the impingement events, for example in the readout period. For this, a further count of the impingement event counter, which initializes at the start of the readout period, that is to say was set to zero, can be incremented by one respectively as soon as a pulse is detected in the second impingement event signal.

One advantage of these embodiments is that with an activated coincidence identification, i.e. with an established connection of the respective further impingement event signal to the logic unit by way of the respective switching unit, the absolute impingement events for different threshold values can be determined simultaneously. Image data can be improved from the information about the absolute impingement events together with the information about the coincidence events in the same readout period.

According to at least one further embodiment, the electronic circuit is designed as an ASIC (application-specific integrated circuit) or as another integrated circuit.

Various types of integrated circuits are known in digital circuit technology which are conceivable for this application, in particular an ASIC can be used, but also other types of circuit.

Other forms of integrated circuits are also conceivable for this embodiment. The ASIC or the integrated circuit can include all of the presented components of the electronic circuit, i.e. in particular the active surface, the comparator, the further comparator, the counter, the further counter, the register, the further register and the at least one circuit part, or include only a subset in a modular structure if these components are provided in the corresponding embodiments.

This embodiment has the advantage of a compact construction with appropriately high performance. The high demands placed on the number of detector pixels and the speed of data processing can thus be met better.

According to at least one embodiment, the electronic circuit includes a switchover unit which is configured to provide the coincidence counter with optionally either the first impingement event signal or the coincidence signal.

In other words, the switchover unit makes it possible to count with the coincidence counter either coincidence events or, alternatively, the impingement events via the first impingement event signal. The impingement events from the first impingement event signal can then be available in the coincidence counter in addition to the impingement events from the second impingement event signal in the impingement event counter for the same readout period.

One advantage of these embodiments is that if the switchover unit provides the first impingement event signal at the coincidence counter, for example the number of impingement events of X-ray photons with different energy thresholds in the case of differing threshold values can be simultaneously captured. Alternatively, the redundancy of the system can be increased in the case of identical threshold values.

According to at least one embodiment, the logic circuit includes an OR gate which is connected at the input side to the at least one further impingement event signal and which is configured to provide a neighboring event signal at an output of the OR gate, and an AND gate which is connected at the input side to the first impingement event signal and the neighboring event signal and which is configured to provide the coincidence signal at an output of the AND gate.

In other words, a test for a simultaneous occurrence of pulses on the first impingement event signal and the at least one further impingement event signal can be implemented by way of the described arrangement of the OR gate and the AND gate. The OR gate can include one input respectively for each of the at least one further impingement event signals and can evaluate the digital signals. The result can be provided at the output of the OR gate as a neighboring event signal. A logical one on the neighboring event signal can therefore signify that one of the at least one further detector pixels has detected an impingement event. At the AND gate the information of the at least one further impingement event signal can be logically linked to that of the first impingement event signal.

The use of OR gates and AND gates is particularly advantageous since the corresponding circuits achieve the necessary results quickly and reliably.

The terms AND gate and OR gate can be understood, in particular, functionally, that is to say it does not necessarily have to be one single gate respectively, instead it can also be a design with other types of gate, in particular with NOR gates or NAND gates. In other words, an AND gate can also be referred to as an AND circuit and an OR gate can be referred to as an OR circuit.

However, it is also possible to use other logical circuits which have the same function, in particular truth tables, as the combination of the OR gate and the AND gate.

According to one or more example embodiments, a photon counting X-ray detector for a CT system is disclosed, which includes an inventive electronic circuit.

Further designs of the inventive X-ray detector follow directly from the different embodiments of the inventive electronic circuit. In particular individual features and corresponding explanations as well as advantages in respect of the various embodiments relating to the inventive method may be transferred analogously to corresponding embodiments of the inventive X-ray detector.

According to one or more example embodiments, a CT system is disclosed which includes an X-ray tube for emitting X-ray photons, an inventive photon counting X-ray detector and a data processing system which is configured to generate CT image data as a function of the coincidence count.

Further embodiments of the inventive CT system follow directly from the different embodiments of the inventive electronic circuit. In particular, individual features and corresponding explanations as well as advantages in respect of the various embodiments relating to the inventive method may be transferred analogously to corresponding embodiments of the inventive CT system.

According to one or more example embodiments, a method for capturing coincidence events of a CT system, which has a photon counting X-ray detector which includes a detector pixel array, is disclosed. A first impingement event signal is provided as a function of an energy captured via a detector pixel of the detector pixel array. In addition, for each further detector pixel of at least one further detector pixel of the detector pixel array, a further impingement event signal is provided as a function of an energy captured via the respective further detector pixel. In addition, the first impingement event signal is compared via a logic circuit, in particular a logic circuit of the X-ray detector, with the at least one further impingement event signal and a coincidence signal is provided as a function of a result of the comparison. A coincidence count is incremented as a function of the coincidence signal. In a first operating mode of the CT system, the logic circuit is provided at the input side with each further impingement event signal of the at least one further impingement event signal. In a second operating mode of the CT system, at least one further impingement event signal of the at least one further impingement event signal is disconnected from the logic circuit.

According to at least one embodiment of the method, a final coincidence count of the coincidence counter is determined at the end of a specified readout period and CT image data is generated as a function of the final coincidence count.

According to at least one embodiment of the method, a number of impingement events is determined during the readout period as a function of the energy captured via the detector pixel and the CT image data is generated as a function of the final coincidence count and the number of impingement events during the readout period.

Further embodiments of the inventive method follow directly from the various embodiments of the inventive electronic circuit, and vice versa. In particular, individual features and corresponding explanations as well as advantages in respect of the various embodiments relating to the inventive electronic circuit may be transferred analogously to corresponding embodiments of the inventive method. In particular, the inventive electronic circuit is embodied or programed to carry out an inventive method. In particular, the inventive electronic circuit carries out the inventive method.

Further features and combinations of features of the invention result from the figures and their description as well as from the claims. In particular, further embodiments of the invention do not necessarily have to include all features of one of the claims. Further embodiments of the inventions can have features or combinations of features which are not mentioned in the claims.

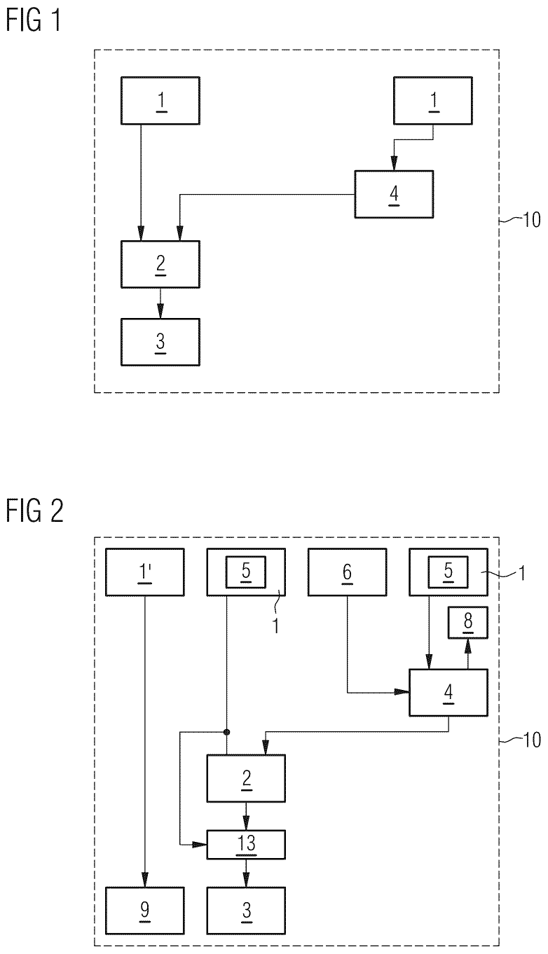

FIG. 1 shows a block diagram of an inventive electronic circuit 10 for capturing coincidence events of a computed tomography system, CT system, which has a photon counting X-ray detector which includes a detector pixel array.

The electronic circuit 10 includes a first capturing unit 1 for a detector pixel 11 and a further capturing unit 1 for a further detector pixel 12 of the at least one further detector pixel 12. The number of the at least one further detector pixel 12 is equal to one, by way of example, in this figure for a simpler representation. This merely serves the underlying mode of operation and does not represent a limitation of the invention. In particular, the number of the at least one further detector pixel 12 can be equal to or greater than one.

The first capturing unit 1 is configured to provide a first impingement event signal as a function of an energy captured via the detector pixel 11. For this, the first capturing unit 1 is connected, in particular, to the active surface of the detector pixel 11 which can capture the energy of the incident X-ray photons of the detector pixel 11. For simpler representation this active surface is not included in FIG. 1 and the following figures. The same applies to the further capturing unit 1 and the second capturing unit 1′.

The first capturing unit 1 is connected at the output side to a logic circuit 2. The further capturing unit 1 is connected at the output side to a switching unit 4. The switching unit 4 is connected at the output side to the logic circuit 2. In other words, the further capturing unit 1 is connected at the output side to the logic circuit 2 via the switching unit 4. The logic circuit 2 therefore receives the first impingement event signal directly and the further impingement event signal via the switching unit 4.

The switching unit 4 is configured to optionally disconnect or establish the connection between the further capturing unit 1 and the logic circuit 2, so optionally the further impingement event signal is transferred or not transferred to the logic circuit 2. The logic circuit 2 can carry out a comparison logic of the two impingement event signals, in particular the first impingement event signal and the further impingement event signal. For example, with a disconnected connection, the switching unit 4 can transmit a signal in the form of a logic zero, a signal in the form of a logic one or another signal in the form of an open state, for example high-resistance or low-resistance, to the logic circuit 2. As a result, a state which does not consume current is implemented which, depending on the logic family used, can have different embodiments. The result of the comparison logic can be transmitted as a coincidence signal from the logic circuit 2 to a coincidence counter 3.

The logic circuit 2 can, for example for the represented case in which the number of the at least one further detector pixel 12 is equal to 1, include an AND gate 16 or be composed of an AND gate. With the simultaneous occurrence of a pulse on the first impingement event signal and on the further impingement event signal and when the switching unit 4 is switched on, this can likewise produce a pulse on the coincidence signal owing to the AND gate 16 in the logic circuit 2.

The coincidence counter 3 can capture a coincidence event which occurs in the form of a pulse in the coincidence signal and with each capture of a further pulse, increase a coincidence count by one. As soon as the switching unit 4 disconnects the connection of the further impingement event signal from the logic circuit 2, the coincidence signal in the represented example, in which the number of the at least one further detector pixel 12 is equal to one, can be equal to zero.

In one exemplary embodiment, the function of the switching unit 4 can be understood as an activation or deactivation of a coincidence identification. When the switching unit 4 is switched on the coincidence counter 3 can capture the number of coincidence events between the detector pixel 11 and the further detector pixel 12. When the switching unit 4 is switched off it possible for the coincidence counter 3 to not capture any impingement events.

The first capturing unit 1 can include, for example, a comparator 5. The comparator 5 can receive, for example, the analog detector signal of the detector pixel 11 at the input side and compare the value with a predefined threshold value. The comparator 5 can output a digital pulse if the predefined threshold value is reached or exceeded. This pulse is then transmitted as a first impingement event signal to the logic circuit 2.

FIG. 2 shows a further exemplary embodiment of the inventive electronic circuit 10, which is based on the exemplary embodiment of FIG. 1. It shows the first capturing unit 1, the further capturing unit 1 and a second capturing unit 1′. The exemplary embodiment shown in FIG. 2 can have all features of FIG. 1. In particular, FIG. 2 also shows an example in which the number of the at least one further detector pixel 12 is equal to one.

The first capturing unit 1 is connected to the logic circuit 2 and, for example, to a switchover unit 13. As above, the further capturing unit 1 is connected to the logic circuit 2 via the switching unit 4.

A control circuit 6 can control the switching unit 4, i.e., for example, change a switching state of the switching unit 4. For example, an alternative signal 8, for example a constant ground potential, can likewise be connected to the switching unit 4. The control circuit 6 can actuate, for example, the switching unit 4 accordingly so the switching unit 4 optionally applies the further impingement event signal or the alternative signal 8 at the output of the switching unit 4. This output is connected to the logic circuit 2. The switching unit 4 can also include, in particular, a multiplexer 7. The multiplexer 7 can optionally transmit one of the signals connected at the input side, i.e. the further impingement event signal or the alternative signal 8, to the logic circuit 2.

The output of the logic circuit 2 is connected to an input of the switchover unit 13 and the output of the switchover unit 13 to the coincidence counter 3. In an exemplary embodiment, the switchover unit 13 can optionally output the coincidence signal of the logic circuit 2 or the first impingement event signal at its output. This output is connected to the coincidence counter 3.

The further capturing unit 1 can include a further comparator 5. This further comparator 5 can behave analogously to the comparator 5. The further comparator 5 can receive, for example, the analog detector signal of the one further detector pixel 12 respectively and compare it with a predefined further threshold value. The further comparator 5 can output a digital pulse if the predefined further threshold value is reached or exceeded. This pulse is then transmitted as a further impingement event signal to the switching unit 4.

The second capturing unit 1′ is connected to an impingement event counter 9. The impingement event counter 9 can count, for example, the impingement events of the detector pixel 11. The second capturing unit 1′ can likewise include a second comparator 5. The second comparator 5 can compare the analog detector signal of the detector pixel 11 with a predefined second threshold value and can output a pulse if the predefined second threshold value is reached or exceeded. In particular, the predefined first threshold value can differ from the predefined second threshold value and thereby the two comparators, the first and the second comparator, can output different signals even if they receive the same detector signal at the input side.

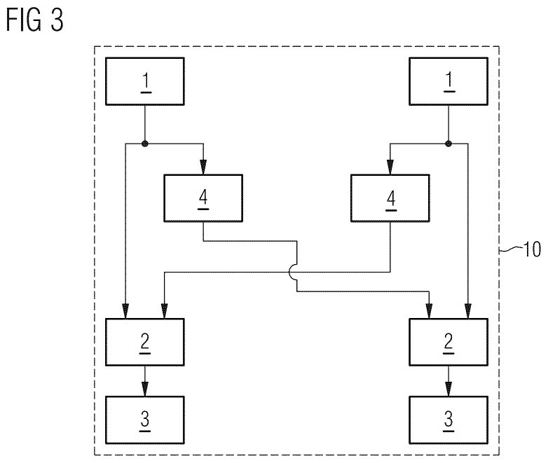

FIG. 3 shows a block diagram of an exemplary embodiment in which the electronic circuit 10 described in respect of FIG. 1 is reciprocally constructed. For example the first capturing unit 1 is connected to a further logic circuit 2 via a further switching unit 4. The detector pixel 11 can therefore receive the further impingement event signal respectively from a further detector pixel 12 respectively at the logic circuit 2, just as, vice versa, the one further detector pixel 12 respectively can receive the first impingement event signal of the detector pixel 11 at the respective further logic circuit 2.

For simpler representation the number of the at least one further detector pixel 12 has also been chosen as one in this block diagram.

An exemplary relationship between two detector pixels 11, 12 for coincidence identification can be seen in the representation in FIG. 3. In an exemplary embodiment, two detector pixels 11, 12 can have one data line respectively, which can be switched off, in each direction, in particular they can have one data line respectively, which can be switched off, in the direction of the other detector pixel 11, 12 respectively and one data line, which can be switched off, away from the other detector pixel 11, 12 respectively.

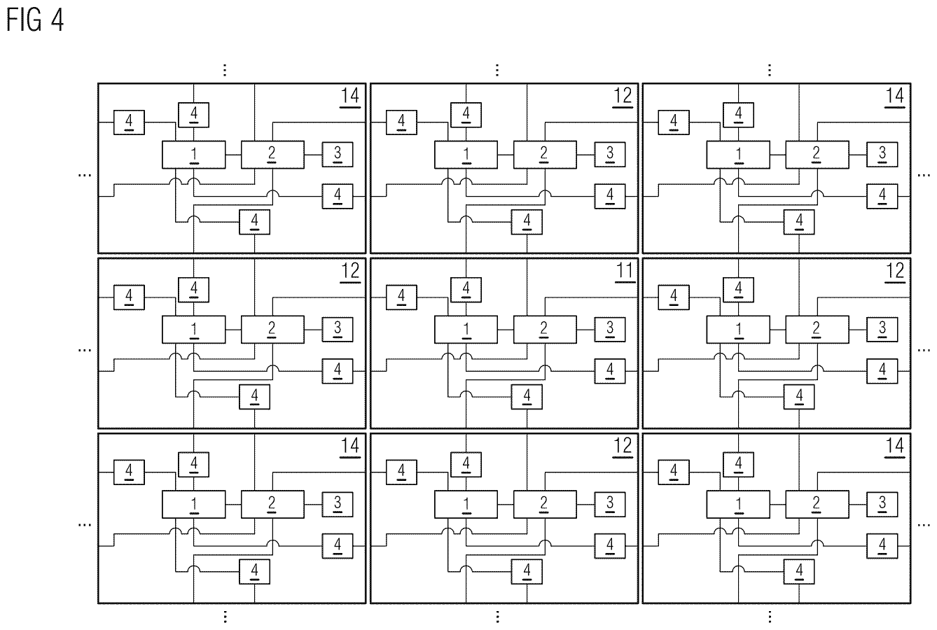

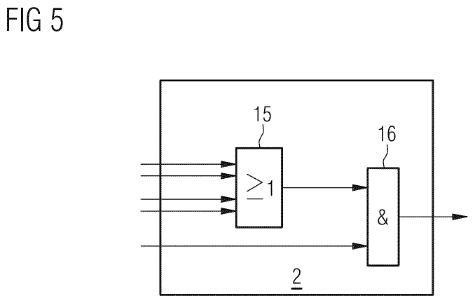

FIG. 4 shows a block diagram of a further exemplary embodiment of an inventive electronic circuit 10. The represented electronic circuit 10 can have the same features of the electronic circuit 10 represented in the other figures. In particular, the electronic circuit 10 can be distributed among a plurality of detector pixels 11, 12 in this representation.

Centrally, FIG. 4 shows a first number of components of the electronic circuit 10, which can be associated with the detector pixel 11. In this representation, the number of the at least one further detector pixel 12 is given, by way of example, as four. This thereby produces, for example, the above-described 4-neighborhood. In addition, a second number of components of the electronic circuit 10 is represented, which can be associated with the four further detector pixels 12 respectively.

In an exemplary embodiment, the first number of components includes the first capturing unit 1, the logic circuit 2, the coincidence counter 3 and four further switching units 4, which respectively connect the first impingement event signal to the adjacent further detector pixel 12. The respective further impingement event signals of the four further detector pixels 12 can be connected to the logic circuit 2 of the detector pixel 11 via the respective switching units 4.

FIG. 4 also represents, by way of example, four diagonal detector pixels 14. In an exemplary embodiment, these diagonal detector pixels do not have a direct connection for the purpose of coincidence identification to the detector pixel 11. In another exemplary embodiment, it is also conceivable to expand the presented system to the 8-neighborhood, with it then being possible for the diagonal detector pixels 14 and the further detector pixels 12 to be connected to the detector pixel 11.

The three points represented in each direction show the continuation of this arrangement. The connections can be analogously constructed, by way of example beyond the detector pixel array. It should be observed in this connection that those detector pixels 11 which are located at the boundary of the detector pixel array can have a reduced number of neighbors. If the detector pixel 11 is located at the boundary and not at a corner, the number of further detector pixels 12 is reduced to three, the number of diagonal detector pixels 14 to two, the 8-neighborhood to five. If the detector pixel 11 is located in a corner, the number of further detector pixels 12 is reduced to two, the number of diagonal detector pixels 14 to one, the 8-neighborhood to three.

The number of impingement event signals, which, by way of example, is necessary for capturing coincidence events in the case of a CT system with a typical number of detector pixels of greater than 1 million, also becomes clear from the representation. These can be individually disconnected, as described in the invention, from the at least one further detector pixel using the large number of switching units 4 and the large number of further switching units 4.

FIG. 5 shows the logic circuit 2 of a further exemplary embodiment of the inventive electronic circuit 10. The logic circuit 2 can include, for example, an OR gate 15 and an AND gate 16.

The OR gate 15 can receive the at least one further impingement event signal at the input side. The represented example shows a number of four for the at least one further detector pixels 12 and accordingly four further impingement event signals at the inputs of the OR gate 15. Similarly, in another exemplary embodiment, the number of the at least one further detector pixel 12 can be greater, for example eight for the case where the diagonal detector pixels 14 are incorporated in the coincidence identification, or also smaller. The OR gate 15 would then accordingly receive more than four further impingement event signals at the input side.

The OR gate 15 can provide a neighboring event signal at an output. In the represented example, the neighboring event signal is connected at the input side to the AND gate. For the case where the number of the at least one further detector pixel is equal to one, the OR gate 15 can be omitted, in particular the further impingement event signal is then identical to the neighboring event signal.

The AND gate 16 receives the first impingement event signal and the neighboring event signal at the input side. The AND gate 16 can thus provide the coincidence signal at the output.

In particular, the arrangement of the OR gate 15 and the AND gate 16 can also be inverted to a certain extent, i.e. the order of the connections can be swapped. For example, the number of AND gates 16 can correspond to the number of the at least one further impingement event signal. Each AND gate 16 can then be connected at the input side to one of the at least one further impingement event signals respectively. In addition, each AND gate 16 can be provided with the first impingement event signal at the input side. The outputs of the AND gate 16 can then be connected at the input side to the OR gate 15. The output of the OR gate 15 corresponds in this exemplary embodiment to the coincidence signal. Other embodiments of the logic circuit 2 are also possible.

Independent of the grammatical term usage, individuals with male, female or other gender identities are included within the term.

It is pointed out once again in conclusion that the method described in detail above, as well as the magnetic resonance apparatus shown, merely involve exemplary embodiments, which can be modified by the person skilled in the art in a very wide variety of ways without departing from the field of the invention. Furthermore the use of the indefinite article “a” or “an” does not exclude the features concerned also being able to be present a number of times. Likewise the terms “unit” and “element” do not exclude the components concerned consisting of a number of interacting part components, which where necessary can also be spatially distributed. Independent of the grammatical term usage of a specific person-related term, individuals with male, female or other gender identities should be included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in order, the reverse depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. An electronic circuit configured to capture coincidence events of a computed tomography (CT) system including a photon counting X-ray detector having a detector pixel array, the electronic circuit comprising:
a first capturing unit configured to provide a first impingement event signal as a function of an energy captured via a detector pixel of the detector pixel array; and
for each of at least one further detector pixel of the detector pixel array,
a further capturing unit, the further capturing unit configured to provide a further impingement event signal as a function of an energy captured via the respective further detector pixel;
a logic circuit configured to compare the first impingement event signal with the at least one further impingement event signal and to provide a coincidence signal as a function of a result of the comparison;
a coincidence counter configured to increment a coincidence count of the coincidence counter as a function of the coincidence signal; and
for each of the at least one further detector pixel of the detector pixel array,
a switching unit, the switching unit configured to disconnect the further impingement event signal from the logic circuit.

2. The electronic circuit of claim 1, wherein at least one of,
the first capturing unit includes a comparator, or
the at least one further capturing unit includes one further comparator, respectively.

3. The electronic circuit of claim 1, wherein
each further detector pixel of the at least one further detector pixel adjoins the detector pixel, or
each further detector pixel of the at least one further detector pixel is in a predefined neighborhood of the detector pixel.

4. The electronic circuit of claim 1, further comprising:
a control circuit configured to control a switching state of the at least one switching unit.

5. The electronic circuit of claim 1, wherein the switching unit includes a multiplexer configured to provide the logic circuit with an alternative signal as an alternative to the further impingement event signal of the respective at least one further detector pixel.

6. The electronic circuit of claim 1, wherein the switching unit is configured to provide the logic circuit with an alternative signal or a constant reference potential as an alternative to the further impingement event signal of the respective at least one further detector pixel.

7. The electronic circuit of claim 1, further comprising:
a second capturing unit configured to provide a second impingement event signal as a function of the energy captured via the detector pixel; and
an impingement event counter configured to increment a count of the impingement event counter as a function of the second impingement event signal.

8. The electronic circuit of claim 1, wherein the electronic circuit is an ASIC or an integrated circuit.

9. The electronic circuit of claim 1, further comprising:
a switchover unit configured to provide the coincidence counter with the first impingement event signal or the coincidence signal.

10. The electronic circuit of claim 1, wherein the logic circuit includes,
an OR gate configured to receive the further impingement event signal and configured to provide a neighboring event signal at an output of the OR gate, and
an AND gate configured to receive the first impingement event signal and the neighboring event signal and configured to provide the coincidence signal at an output of the AND gate.

11. A photon counting X-ray detector for a CT system, comprising:
the electronic circuit of claim 1.

12. A CT system comprising:
an X-ray tube configured to emit X-ray photons;
the photon counting X-ray detector of claim 11; and
a data processing system configured to generate CT image data as a function of the coincidence count of the coincidence counter.

13. A method for capturing coincidence events of a CT system including a photon counting X-ray detector having a detector pixel array, the method comprising:
providing a first impingement event signal as a function of an energy captured via a detector pixel of the detector pixel array; and
for each further detector pixel of at least one further detector pixel of the detector pixel array,
providing a further impingement event signal a function of an energy captured via the respective further detector pixel,
comparing the first impingement event signal compared via a logic circuit with the at least one further impingement event signal, and
providing a coincidence signal based on the comparing,
incrementing a coincidence count as a function of the coincidence signal;
in a first operating mode of the CT system of the logic circuit, providing each of the at least one further impingement event signals at an input side; and
in a second operating mode of the CT system, disconnecting at least one further impingement event signal of the at least one further impingement event signal from the logic circuit.

14. The method of claim 13, further comprising:
determining a final coincidence count at an end of a specified readout period; and
generating CT image data as a function of the final coincidence count.

15. The method of claim 14, further comprising:
determining a number of impingement events during the readout period as a function of the energy captured via the detector pixel; and
the generating generates the CT image data as a function of the final coincidence count and the number of impingement events during the readout period.

16. The electronic circuit of claim 4, wherein the switching unit is configured to provide the logic circuit with an alternative signal or a constant reference potential as an alternative to the further impingement event signal of the respective at least one further detector pixel.

17. The electronic circuit of claim 16, further comprising:
a second capturing unit configured to provide a second impingement event signal as a function of the energy captured via the detector pixel; and
an impingement event counter configured to increment a count of the impingement event counter as a function of the second impingement event signal.

18. The electronic circuit of claim 17, wherein the electronic circuit is an ASIC or an integrated circuit.

19. The electronic circuit of claim 18, further comprising:
a switchover unit configured to provide the coincidence counter with the first impingement event signal or the coincidence signal.

20. The electronic circuit of claim 19, wherein the logic circuit includes,
an OR gate configured to receive the further impingement event signal and configured to provide a neighboring event signal at an output of the OR gate, and
an AND gate configured to receive the first impingement event signal and the neighboring event signal and configured to provide the coincidence signal at an output of the AND gate.
